(19) United States
(12) Patent Application Publication
(10) Pub. No.: US 2011/0257532 A1
(43) Pub. Date: Oct. 20, 2011

(54) ULTRASONIC PROBE AND METHOD OF PREPARING ULTRASONIC PROBE

(75) Inventor: Takayuki Sasaki, Tokyo (JP)

(73) Assignee: Konica Minolta Medical & Graphic, Inc., Hino-shi, Tokyo (JP)

(21) Appl. No.: 13/141,619

(22) PCT Filed: Dec. 11, 2009
(86) PCT No.: PCT/JP2009/070741

§ 371 Date: Jun. 22, 2011

(30) Foreign Application Priority Data
Dec. 25, 2008 (JP) 2008-329277

(51) Int. Cl. A61B 8/00; H01L 41/22

(52) U.S. Cl. 600/459; 29/25.35

(57) ABSTRACT

Provided is an ultrasonic probe possessing signal lines for which generation of the acoustic crosstalk between piezoelectric elements to each other constituting a piezoelectric element array, and also provided is a method of preparing the ultrasonic probe. Disclosed is an ultrasonic probe comprising a first piezoelectric element array in which first piezoelectric elements each as a both-sided electrodes-providing piezoelectric element are two-dimensionally arrayed; a second piezoelectric element array layered on the first piezoelectric element array; an acoustic separation section provided between the first piezoelectric elements to each other, arrayed in the first piezoelectric element array; and a signal line possessing a conductive layer coated on an outer circumferential surface of a core material and the core material having an acoustic impedance nearly equal to another acoustic impedance of the acoustic separation section, the signal line connected to the second electrode by passing through the acoustic separation section.

### TECHNICAL FIELD

The present invention relates to an ultrasonic probe suitably used for ultrasonic apparatuses such as an ultrasonic diagnosis apparatus and so forth.

### BACKGROUND

The ultrasonic diagnosis apparatus has been widely used for cross-sectional image-visualization of blood flow in the body and body tissues. The ultrasonic diagnosis apparatus transmits ultrasonic waves to body tissues, employing an ultrasonic probe, and receives reflection echo produced by the tissue interface impedance difference by narrowing the ultrasonic waves down to a fine beam to obtain ultrasonic wave images via image formation of tissues in the body.

The ultrasonic diagnosis apparatus conventionally places an array type probe in which piezoelectric elements are one-dimensionally arrayed, in an ultrasonic probe, and two-dimensionally scans with ultrasonic waves to form body cross-sectional images.

Appearance of a 2D array type probe equipped with a piezoelectric element army in which piezoelectric elements are two-dimensionally arrayed has recently been seen. Specifically, in order to expand the reception range, proposed is an ultrasonic probe in which the first piezoelectric element array to receive fundamental waves and the second piezoelectric element array to receive harmonic waves among waves reflected from the object (refer to Patent Document 1).

It is difficult to connect signal lines to piezoelectric elements constituting each piezoelectric element array, when two piezoelectric element arrays of the first piezoelectric element army and the second piezoelectric element array have been provided. In contrast, in order to reduce the space where signal lines are occupied, proposed is a technique by which the signal lines passing through are wired in an acoustic damping member provided on the back side of a piezoelectric element (refer to Patent Document 2).

### PRIOR ART DOCUMENT

### Patent Document

Patent Document 1: Japanese Patent Open to Public Inspection (O.P.I.) Publication No. 11-276478
Patent Document 2: Japanese Patent O.P.I. Publication No. 2000-166923

### DISCLOSURE OF THE INVENTION

### Problems to be Solved by the Invention

When using the technique disclosed in Patent Document 2, since an acoustic impedance of the signal line is larger than another impedance of the acoustic damping member, ultrasonic waves are reflected in the region between the acoustic damping member and the signal line, resulting in generation of an acoustic crosstalk to a piezoelectric element.

Specifically in the technique disclosed in Patent Document 1, when signal lines from piezoelectric elements constituting the second piezoelectric element array are designed to be wired by passing through the interface between piezoelectric elements of the first piezoelectric element array to each other, a signal line of high acoustic impedance is to be present between acoustic separation materials each between piezoelectric elements constituting the first piezoelectric element array to each other, whereby crosstalk is generated to the first piezoelectric element, resulting in deterioration of performance of the ultrasonic probe.

It is an object of the present invention to provide an ultrasonic probe possessing signal lines for which generation of the acoustic crosstalk between piezoelectric elements to each other constituting a piezoelectric element array is inhibited, and a method of preparing the ultrasonic probe.

### Means to Solve the Problems

The foregoing object is accomplished by the following structures.

(Structure 1) An ultrasonic probe comprising a first piezoelectric element array in which first piezoelectric elements each as a both-sided electrodes-providing piezoelectric element are two-dimensionally arrayed; a second piezoelectric element array layered on the first piezoelectric element array, in which second piezoelectric elements are two-dimensionally arrayed, a first electrode is placed on a second piezoelectric element surface on an opposite side of the first piezoelectric element array, and a second electrode is placed on another second piezoelectric element surface on a side of the first piezoelectric element array, an acoustic separation section provided between the first piezoelectric elements to each other, arrayed in the first piezoelectric element array; and a signal line comprising a conductive layer coated on an outer circumferential surface of a core material and the core material having an acoustic impedance nearly equal to another acoustic impedance of the acoustic separation section, the signal line connected to the second electrode by passing through the acoustic separation section.

(Structure 2) The ultrasonic probe of Structure 1, wherein an area of each of the first piezoelectric elements to which a voltage is applied is different from an area of each of the second piezoelectric elements to which a voltage is applied.

(Structure 3) The ultrasonic probe of Structure 1 or 2, wherein the number of the first piezoelectric elements arrayed in the first piezoelectric element array is different from the number of the second piezoelectric elements arrayed in the second piezoelectric element army.

(Structure 4) A method of preparing an ultrasonic probe, comprising the steps of forming a through-hole in an acoustic separation section provided between the first piezoelectric elements to each other in a first piezoelectric element array in which first piezoelectric elements each as a both-sided electrodes-providing piezoelectric element are two-dimensionally arrayed; forming a conductive layer on a through-hole inner circumferential surface so as to form a through-hole inner spacing; and filling a core material having an acoustic impedance nearly equal to another acoustic impedance of the acoustic separation section in the through-hole inner spacing.

### Effect of the Invention

Provided can be an ultrasonic probe possessing signal lines for which generation of the acoustic crosstalk between piezoelectric elements to each other constituting a piezoelectric element array is inhibited without producing cost rises while maintaining the ultrasonic probe in size, and a method of preparing the ultrasonic probe can also be provided.

### BRIEF DESCRIPTION OF THE DRAWINGS

FIG. 1 is a diagram showing the external view configuration of ultrasonic diagnosis apparatus H in an embodiment.

FIG. 2 is a block diagram showing electrical constituent parts of ultrasonic diagnosis apparatus H in an embodiment.

FIG. 3 is a cross-sectional diagram showing constituent parts of ultrasonic probe 2 installed in an ultrasonic diagnosis apparatus in an embodiment.

FIG. 4 shows a preparation flowchart in a method of preparing ultrasonic probe 2.

FIGS. 5a, 5b, 5c and 5d each show a schematic diagram in a method of preparing organic piezoelectric element array 5.

FIGS. 6a and 6b each show a schematic diagram of acoustic damping member 23.

FIGS. 7a and 7b each show a schematic diagram of acoustic matching layer 31.

FIGS. 8a and 8b each show a schematic diagram of a work body in which flat plate-shaped double-sided electrodes-providing inorganic piezoelectric element 50 provided on both surfaces of the inorganic piezoelectric element is instilled.

FIGS. 9a and 9b each show a schematic diagram of a work body in which inorganic piezoelectric element 22 is arrayed.

FIGS. 10a and 10b each show a schematic diagram of a work body in which acoustic separation section 24 is formed.

FIGS. 11a and 11b each show a schematic diagram of a work body in which common ground electrode 25 is formed.

FIGS. 12a and 12b each show a schematic diagram of a work body in which acoustic matching layer 26 is formed.

FIGS. 13a and 13b each show a schematic diagram of a bored work body.

FIGS. 14a and 14b each show a schematic diagram of a work body in which photoresist is formed.

FIG. 15 shows a schematic diagram of a process by which metal constituting conductive member 32 is evaporated on the work body.

FIGS. 16a and 16b each show a schematic diagram of a work body from which the metal constituting conductive member 32 on photoresist, together with the photoresist, is removed.

FIGS. 17a and 17b each show a schematic diagram of a work body in which core material 33 is formed in laser processing hole 51.

FIGS. 18a and 18b each show a schematic diagram of a work body possessing a layered organic piezoelectric element array.

FIGS. 19a and 19b each show a schematic diagram of a work body in which acoustic matching layer 27 is formed.

FIG. 20 shows a preparation flowchart for a method of preparing the first piezoelectric element array 4.

FIGS. 21a and 21b each show a schematic diagram of a substrate having a piezoelectric material thereon.

FIGS. 22a and 22b each show a schematic diagram of a work body having been subjected to sandblast processing.

FIGS. 23a and 23b each show a schematic diagram of a work body in which acoustic separation section 24 is formed.

FIGS. 24a and 24b each show a schematic diagram of a work body in which the acoustic separation section is exposed onto both surfaces of the acoustic separation section via polishing.

FIGS. 25a and 25b each show a schematic diagram of a work body in which electrodes 102 and 103 are formed on both surfaces of PZT 201.

### DESCRIPTION OF THE PREFERRED EMBODIMENTS

Next, an embodiment of the present invention will be described referring to figures, but the present invention is not limited thereto. In addition, constituent elements to which the same numerals are attached represent the same constituent elements as to each other, and descriptions thereof are omitted.

### (Each Constituent Element and Operation of Ultrasonic Diagnosis Apparatus and Ultrasonic Probe)

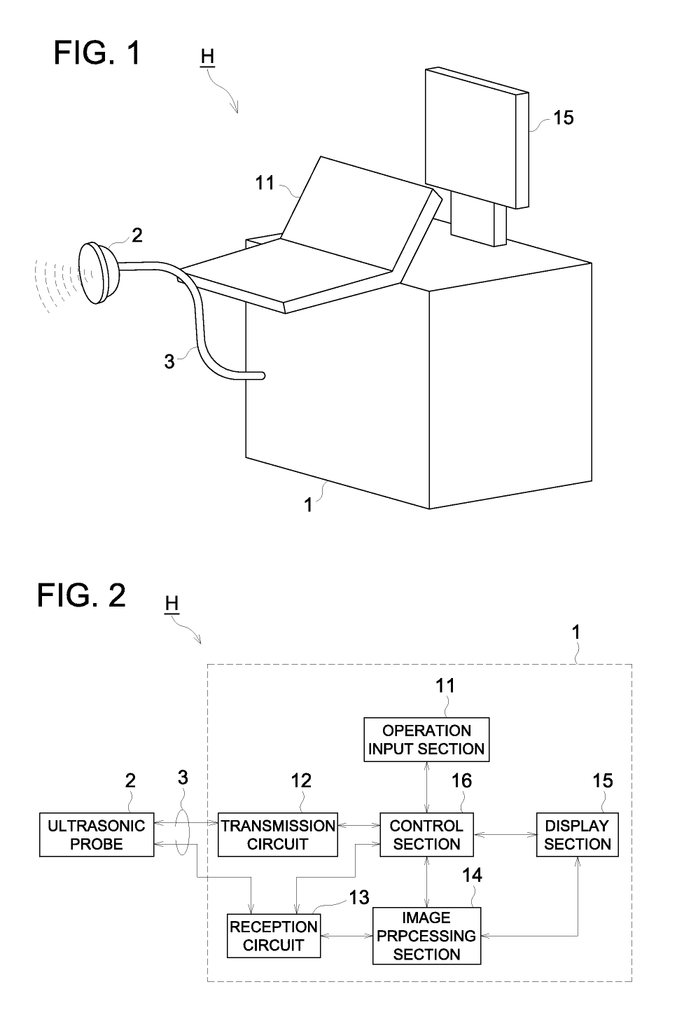

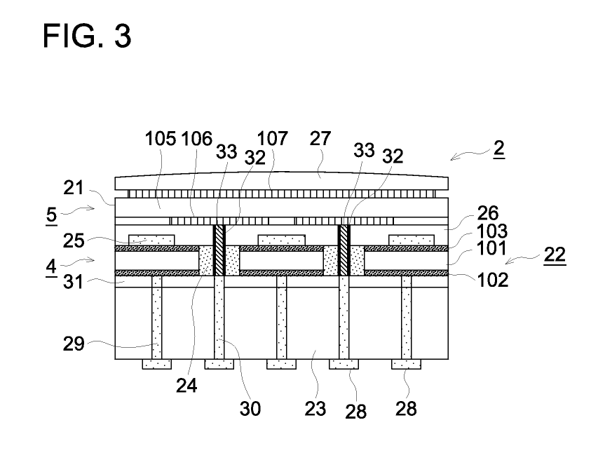

FIG. 1 is a diagram showing the external view configuration of an ultrasonic diagnosis apparatus in an embodiment. FIG. 2 is a block diagram showing electrical constituent parts of the ultrasonic diagnosis apparatus in an embodiment. FIG. 3 is a cross-sectional diagram showing constituent parts of an ultrasonic probe installed in an ultrasonic diagnosis apparatus in an embodiment.

As shown in FIG. 1, ultrasonic diagnosis apparatus H is connected to ultrasonic probe 2 not only to transmit ultrasonic waves (ultrasonic wave signal) to an object such as a body omitted in the figure or the like but also to receive reflected waves (echo, or ultrasonic wave signal) as ultrasonic waves reflected at the object, via cable 3, and transmits ultrasonic waves to ultrasonic probe 2 with respect the object by transmitting signals as electrical signals into ultrasonic probe 2 via cable 3. In addition, the diagnosis apparatus possesses ultrasonic diagnosis apparatus main body 1 to image-visualize the internal situation inside the object as an ultrasonic wave image, based on reception signals as electrical signals produced in ultrasonic probe 2 in response to ultrasonic waves as reflected waves from the inside of the object which are received with ultrasonic probe 2.

As shown in FIG. 2, for example, ultrasonic diagnosis apparatus main body 1 possesses operation input section 11 to input data such as a command to order the diagnosis-start and private information of the object; transmission circuit 12 to make ultrasonic probe 2 to generate ultrasonic waves by supplying the transmission signal as an electrical signal into ultrasonic probe 2 via cable 3; reception circuit 13 to receive the reception signal as an electrical signal from ultrasonic Probe 2 via cable 3; image processing section 14 to produce images (ultrasonic wave images) of the internal situation within the object, based on the reception signal received at reception circuit 13; display section 15 to display images of the internal situation within the object which are produced at image processing section 14; and control section 16 to totally control ultrasonic diagnosis apparatus H by controlling the foregoing operation input section 11, transmission circuit 12, reception circuit 13, image processing section 14 and display section 15, depending on each of functions thereof.

Ultrasonic probe (ultrasonic wave probe) 2 possesses an inorganic piezoelectric element and an organic piezoelectric element. The inorganic piezoelectric element is made of an inorganic piezoelectric material, and can convert signals mutually between electrical signals and ultrasonic wave signals by utilizing a piezoelectric phenomenon. The organic piezoelectric element is made of an organic piezoelectric material, and can convert signals mutually between electrical signals and ultrasonic wave signals by utilizing the piezoelectric phenomenon.

Ultrasonic probe 2 having such a structure can be exemplified, for example, as ultrasonic probe 2 having a structure as shown in FIG. 3.

Ultrasonic probe 2 possesses flat plate-shaped acoustic damping member 23; acoustic matching layer 31 layered on acoustic matching member 23; inorganic piezoelectric element array (the first piezoelectric element army) 4 composed of plural inorganic piezoelectric elements (the first piezoelectric elements) 22 layered to support on one main surface of acoustic damping member 23; acoustic separation section 24 prepared by filling in acoustic separation material in the spacing of inorganic piezoelectric element 22-to-inorganic piezoelectric element 22; common ground electrode 25 layered on each of plural inorganic piezoelectric elements 22; acoustic matching layer 26 layered on common ground electrode 25; organic piezoelectric element array (the second piezoelectric element array) 5 fitted with organic piezoelectric element 21 (the second piezoelectric element) layered on acoustic matching layer 26; acoustic matching layer 27 layered on organic piezoelectric element 21; the first signal line 29 with which conductive pad 28 to receive electrical signals from the outside is connected to the electrode of inorganic piezoelectric element 22; the second signal line 30 with which conductive pad 28 to receive electrical signals from the outside is connected to the electrode of the organic piezoelectric element; and so forth.

Acoustic damping member 23 made of a material to absorb ultrasonic waves absorbs ultrasonic waves radiated in the direction of acoustic damping member 23 from plural inorganic piezoelectric elements 22.

Acoustic matching layer 31 has a medium acoustic impedance between an acoustic impedance of acoustic damping member 23 and another acoustic impedance of each of inorganic piezoelectric elements 22, and the acoustic impedance is matched between acoustic damping member 23 and inorganic piezoelectric elements 22.

Each of inorganic piezoelectric elements 22 possesses electrodes 102 and 103 provided on both surfaces of piezoelectric element 101 made of an inorganic piezoelectric material, which are opposed to each other. Plural inorganic piezoelectric elements 22 are two-dimensionally arrayed to each other, spacing at the predetermined intervals in planar view, and placed on acoustic damping member 23 as the first piezoelectric element array.

Plural inorganic piezoelectric elements 22 may be arrayed so as to receive ultrasonic waves as reflected waves, but in the case of ultrasonic probe 2 and ultrasonic diagnosis apparatus H, they are arranged so as to transmit ultrasonic waves. More specifically, an electrical signal is input to plural inorganic piezoelectric elements 22 from transmission circuit 12 via cable 3, conductive pad 28 and the first signal line 29. This electrical signal is input between electrode 102 and electrode 103 of inorganic piezoelectric elements 22. Plural inorganic piezoelectric elements 22 transmit ultrasonic wave signals by converting this electrical signal into the ultrasonic wave signals.

Acoustic separation section 24 is made of a low acoustic impedance resin having a largely different value of acoustic impedance from that of inorganic piezoelectric element 22 and serves as an acoustic separation material via large difference in acoustic impedance, and has a function of reducing mutual interference of plural inorganic piezoelectric elements 22.

Common ground electrode 25 made of a conductive material is grounded by wiring in an unshown figure, and each of electrodes 103 in inorganic piezoelectric elements 22 is electrically grounded by linearly layering common ground electrode 25 on plural inorganic piezoelectric elements 22, straddling them.

Acoustic matching layer 26 has a medium acoustic impedance between an acoustic impedance of acoustic organic piezoelectric element 21 layered in the postprocessing step and another acoustic impedance of each of inorganic piezoelectric elements 22, and matching the acoustic impedance between organic piezoelectric element 21 and inorganic piezoelectric element 22 is made.

Organic piezoelectric element 21 is a sheet-shaped piezoelectric element possessing piezoelectric element 105 formed from organic piezoelectric material in the form of a flat plate having the predetermined thickness; plural electrodes 106 separated to each other and formed on one main surface of piezoelectric element 105; and electrode 107 evenly formed on the mostly entire surface for the other main surface of piezoelectric element 105.

When plural electrodes 106 are formed on one main surface of piezoelectric element 105 in this way, as to organic piezoelectric element 21, a piezoelectric element composed of one electrode 107, piezoelectric element 105 and electrode 106 can be two-dimensionally arrayed as the second piezoelectric element array, and each of these piezoelectric elements can be individually operated.

Plural piezoelectric elements as organic piezoelectric element 21 do not need to be separated from each other to individually function them like inorganic piezoelectric element 22, and are possible to be formed integrally in the form of a sheet. Accordingly, in a method of preparing organic piezoelectric element 21, there is no step of forming grooves on a plate-shaped body in the form of a sheet made of an organic piezoelectric material, resulting in further simplification of method of preparing organic piezoelectric element 21, whereby organic piezoelectric element 21 can be formed via fewer steps. In addition, since organic piezoelectric element 21 would appear to be composed of plural piezoelectric elements, it may be composed of plural electrodes 106 and plural electrodes which should become each of the pairs in place of electrode 107.

In an example shown in FIG. 3, organic piezoelectric element 21 is layered indirectly on plural inorganic piezoelectric elements 22 via common ground electrode 25 and acoustic matching layer 26 entirely for plural inorganic piezoelectric elements 22. In addition, organic piezoelectric element 21 may be layered on a part of plural inorganic piezoelectric elements 22.

Further, the number of electrodes 106 of organic piezoelectric element 21 (the number of organic piezoelectric elements 21) may be equal to the number of inorganic piezoelectric elements 22, but in the present embodiment, the number of electrodes 106 of organic piezoelectric element 21 may be different from the number of inorganic piezoelectric elements 22. That is, the number of piezoelectric elements as organic piezoelectric elements 21 is different from the number of inorganic piezoelectric elements 22. For this reason, even though the total area of plural inorganic piezoelectric elements 22 is identical to the total area of organic piezoelectric element 21 composed of plural piezoelectric elements, the area occupied by one inorganic piezoelectric element 22 and the area occupied by one piezoelectric element in organic piezoelectric element 21 are possible to be independently designed to be set. Accordingly, inorganic piezoelectric element 22 can be designed in accordance with the specification specified for the inorganic piezoelectric element 22, and at the same time, organic piezoelectric element 21 becomes possible to be designed in accordance with the specification specified for the organic piezoelectric element 21.

Further, the area of electrode 106 of organic piezoelectric element 21 and the area of electrode 102 of inorganic piezoelectric element 22 in addition to the area of electrode 103 of inorganic piezoelectric element 22 can be designed in accordance with the specifications specified for the inorganic piezoelectric element 22 and the organic piezoelectric element 21 by making the area of electrode 106 of organic piezoelectric element 21 to be different from the area of electrode 102 of inorganic piezoelectric element 22 or the area of electrode 103 of inorganic piezoelectric element 22.

The total area of grooves each between inorganic piezoelectric elements 22 is designed to be small by making the area of; for example, one inorganic piezoelectric element 22, and similarly, transmission power of ultrasonic probe 2 can be increased by increasing the total area obtained via addition of the area of each of inorganic piezoelectric elements 22. Further, the area of each piezoelectric element can be designed to be small by increasing the number of piezoelectric elements as organic piezoelectric element 21 to improve reception signal resolution thereof when using organic piezoelectric element 21 for the reception signal.

Organic piezoelectric element 21 may be constituted so as to transmit ultrasonic waves, but in the case of ultrasonic probe 2 and ultrasonic diagnosis apparatus H in the present embodiment, it is constituted so as to receive ultrasonic waves as reflected waves. More specifically, when ultrasonic wave signals as reflected waves are received, and the ultrasonic wave signals are converted into electrical signals, organic piezoelectric element 21 outputs the electrical signals. The electrical signals are output from electrode 106 and electrode 107 of organic piezoelectric element 21. The electrical signals are output to reception circuit 13 via cable 3.

The second signal line 30 is connected to conductive pad 28 receiving electrical signals from the outside, and electrode 106 of organic piezoelectric element 21. That is, the second signal line 30 is connected to conductive pad 28, passing through acoustic matching layer 26, acoustic separation section 24, acoustic matching layer 31 and acoustic damping member 23 from electrode 106 of organic piezoelectric element 21. The portion passing through acoustic damping member 23 and acoustic matching layer 31 from conductive pad 28 has the same structure as that of the first signal line 29 to supply electrical signals into inorganic piezoelectric element 22 from the outside.

The portion passing through acoustic separation section 24 and acoustic matching layer 26 is composed of conductive member 32 and core material 33 having low acoustic impedance. Conductive member 32 has a function of conducting the portion passing through acoustic damping member 23 and acoustic matching layer 31 from conductive pad 28, and electrode 106 of organic piezoelectric element 21. Core material 33 has an acoustic impedance equal to that of acoustic separation 24.

Core material 33 is formed as a cylindrical core material, and conductive member 32 is formed by thinly coating on the circumferential surface of core material 33. Core material 33 preferably has a diameter of 50-60 μm, for example, and conductive member 32 preferably has a thickness of approximately 0.05 μm, for example. When making the thickness to be approximately 0.05 μm, minimized can be reflection of ultrasonic waves at the interface between acoustic separation section 24 and conductive member 32, and at the interface between core material 33 and conductive member 32 by supplying sufficient electrical power to drive organic piezoelectric element 21 with ultrasonic waves, whereby generation of acoustic crosstalk between inorganic piezoelectric elements 22 can be avoided.

A material for each of acoustic separation section 24 and core material 33 is selected in such a way that an acoustic impedance of acoustic separation section 24 and another acoustic impedance of core material 33 are closely identical to each other. Reflection of ultrasonic waves between acoustic separation section 24 and core material 33 can be largely reduced by setting the acoustic impedance values for the two materials to those closely identical to each other, whereby generation of acoustic crosstalk between inorganic piezoelectric elements 22 can be inhibited.

Acoustic matching layer 27 means a member to match an acoustic impedance of organic piezoelectric element 21 with another acoustic impedance of the object. In addition, acoustic matching layer 27 is designed to be swelled in the form of a circular arc, and serves as an acoustic lens converging ultrasonic waves transmitted toward the object.

In the case of ultrasonic wave diagnosis apparatus H having such a structure, when order of diagnosis-start is input from operation input section 11, for example, electrical signals as transmission signals are produced in transmission circuit 12 via controlling of control section 16. The resulting electrical signals as transmission signals are supplied into ultrasonic probe 2 via cable 3. More specifically, the electrical signals as transmission signals are supplied into each of plural inorganic piezoelectric elements 22 in ultrasonic probe 2. The electrical signals as transmission signals are voltage pulses repeated at the predetermined periods, for example. Each of plural inorganic piezoelectric elements 22 is expanded and contracted in the thickness direction of each of them by supplying the electrical signals as transmission signals to conduct ultrasonic wave vibrations in accordance with the electrical signals as transmission signals. Plural inorganic piezoelectric elements 22 radiate ultrasonic waves by way of common ground electrode 25, acoustic matching layer 26, organic piezoelectric element 21 and acoustic matching layer 27 via the ultrasonic wave vibrations. In cases where ultrasonic probe 2 is brought into contact with the object, for example, ultrasonic waves are transmitted into the object from ultrasonic probe 2.

In addition, ultrasonic probe 2 may be used by inserting it inside the object, and may be used by inserting it in the living body cavity, for example.

Ultrasonic waves transmitted into the object are reflected at one interface or plural interfaces having different acoustic impedances inside the object to become ultrasonic waves as reflected waves. The reflected waves include not only the frequency (fundamental frequency of the fundamental waves) component of transmitted ultrasonic waves but also the frequency component of higher harmonic waves in which the fundamental frequency is integrally multiplied. They include, for example, the second harmonic waves for twofold fundamental frequency, the third harmonic waves for threefold fundamental frequency, the fourth harmonic waves for fourfold fundamental frequency and so forth. The ultrasonic waves as reflected waves are received with ultrasonic probe 2. More specifically, ultrasonic waves as reflected waves are received with organic piezoelectric element 21 via acoustic matching layer 27 to convert mechanical vibrations into electrical signals at organic piezoelectric element 21, and taken out as reception signals. The electrical signals taken out as reception signals are received by reception circuit 13 controlled with control section 16 via cable 3.

Herein, as to the above-described, ultrasonic waves are transmitted toward the object from each of inorganic piezoelectric elements 22 in order, and ultrasonic waves reflected at the object are received with organic piezoelectric element 21.

Then, image processing section 14 produces images (ultrasonic wave images) of an internal situation inside the object from a duration from signal transmission to signal reception and reception intensity, based on the reception signal received at reception circuit 13 controlled by control section 16, and display section 15 displays images of internal situation inside the object produced at image processing section 14 controlled by control section 16. In the case of ultrasonic probe 2 and ultrasonic diagnosis apparatus H in the present embodiment, since higher harmonic waves to fundamental waves are received as described above, it becomes possible to form ultrasonic wave images via a harmonic imaging technique. For this reason, ultrasonic probe 2 and ultrasonic diagnosis apparatus H in the present embodiment become capable of providing high quality ultrasonic wave images. Further, since the second higher harmonic waves and the third higher harmonic waves exhibiting considerably large power are received, sharper ultrasonic wave images are possible to be provided.

Further, in the case of ultrasonic probe 2 and ultrasonic diagnosis apparatus H in the present embodiment, plural inorganic piezoelectric elements 22 are designed so as to transmit ultrasonic waves, ultrasonic probe 2 and ultrasonic diagnosis apparatus H can provide larger transmission power at considerably simple structure thereof. Accordingly, ultrasonic probe 2 and ultrasonic diagnosis apparatus H in the present embodiment are suitable for a harmonic imaging technique to transmit fundamental waves at considerably large power in order to obtain higher harmonic echo, and become possible to provide higher quality ultrasonic wave images.

Further, in the case of ultrasonic probe 2 and ultrasonic diagnosis apparatus H in the present embodiment, organic piezoelectric element 21 is designed so as to receive ultrasonic waves as reflected waves. In general, a piezoelectric element made of an inorganic piezoelectric material can receive only about twofold frequency of the fundamental wave frequency, but a piezoelectric element made of an organic piezoelectric material can receive, for example, about fourfold to five fold frequency of fundamental wave frequency, and is suitable for widely expanding the reception frequency range. Since ultrasonic wave signals are received with organic piezoelectric element 21 exhibiting characteristics capable of receiving such ultrasonic waves over the wide frequency range, ultrasonic probe 2 and ultrasonic diagnosis apparatus H in the present embodiment can expand the frequency range widely at considerably simple structure thereof.

In addition, in the above-described, shown is the case where inorganic piezoelectric element 22 and organic piezoelectric element 21 are two-dimensionally arrayed, but they may be one-dimensionally arrayed depending on applications.

### (Method of Preparing Ultrasonic Probe 2)

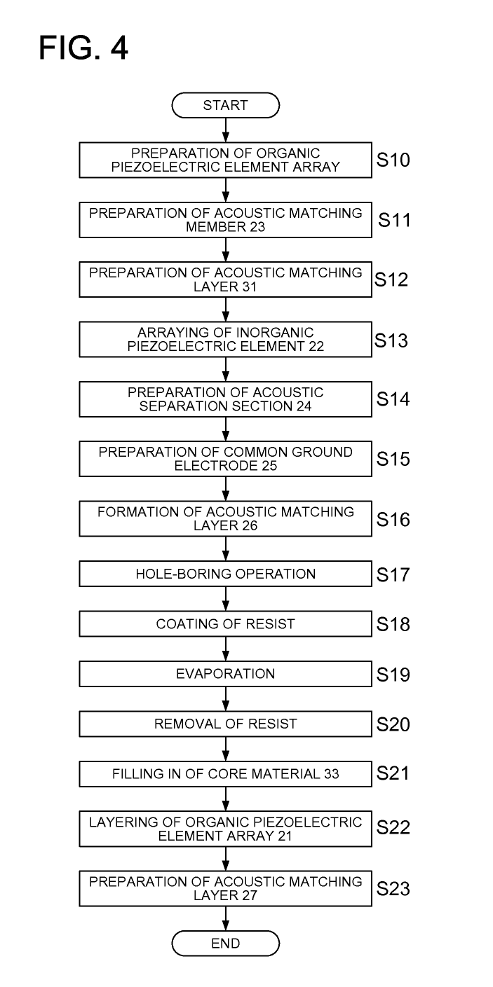

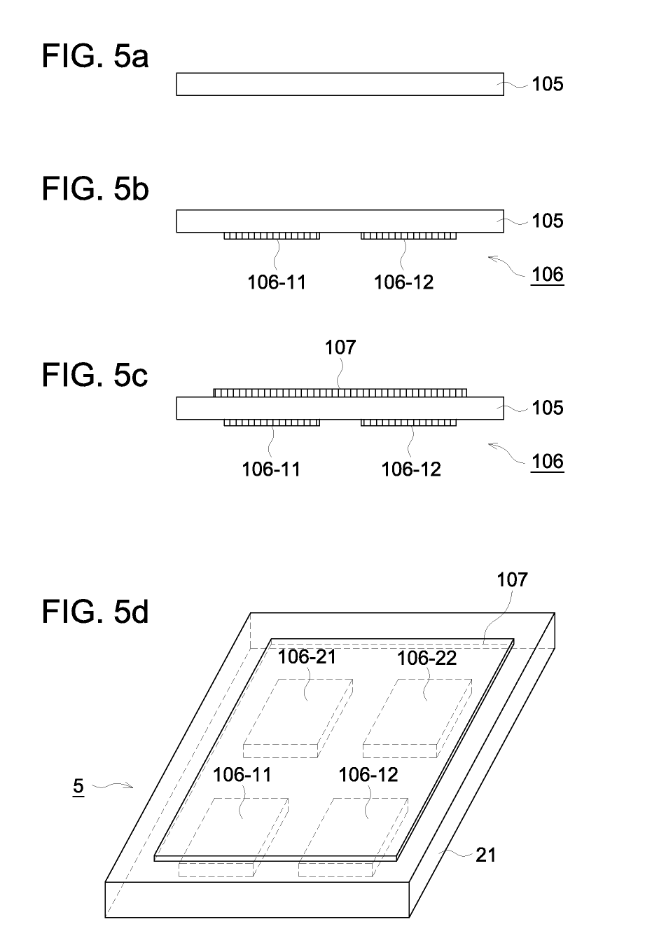

The method of preparing ultrasonic probe 2 will be described referring to FIG. 4, FIGS. 5a-5d, FIGS. 6a and 6b, FIGS. 7a and 7b, FIGS. 8a and 8b, FIGS. 9a and 9b, FIGS. 10a and 10b, FIGS. 11a and 12b, FIGS. 13a and 13b, FIGS. 14a and 14b, FIG. 15, and FIGS. 16a and 16b.

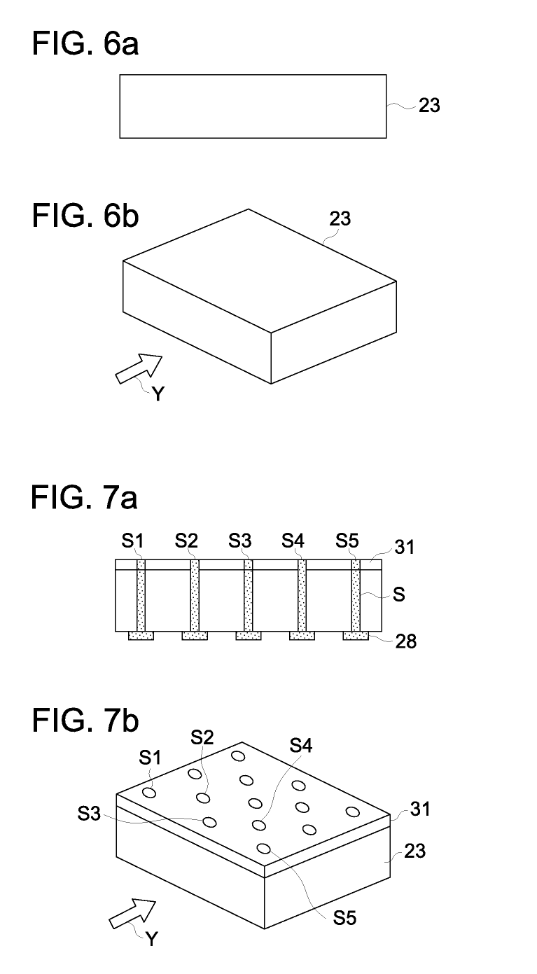

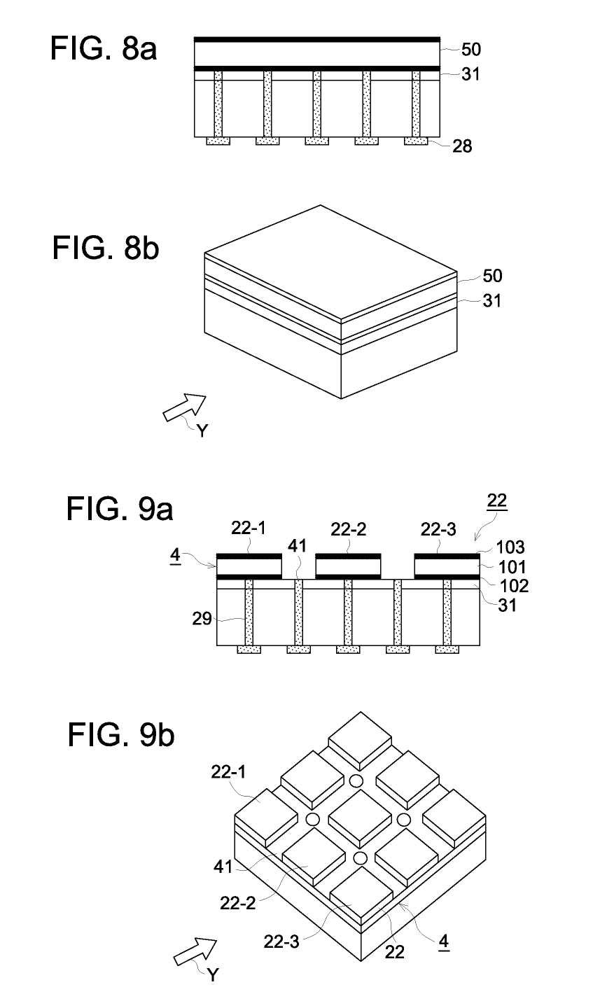

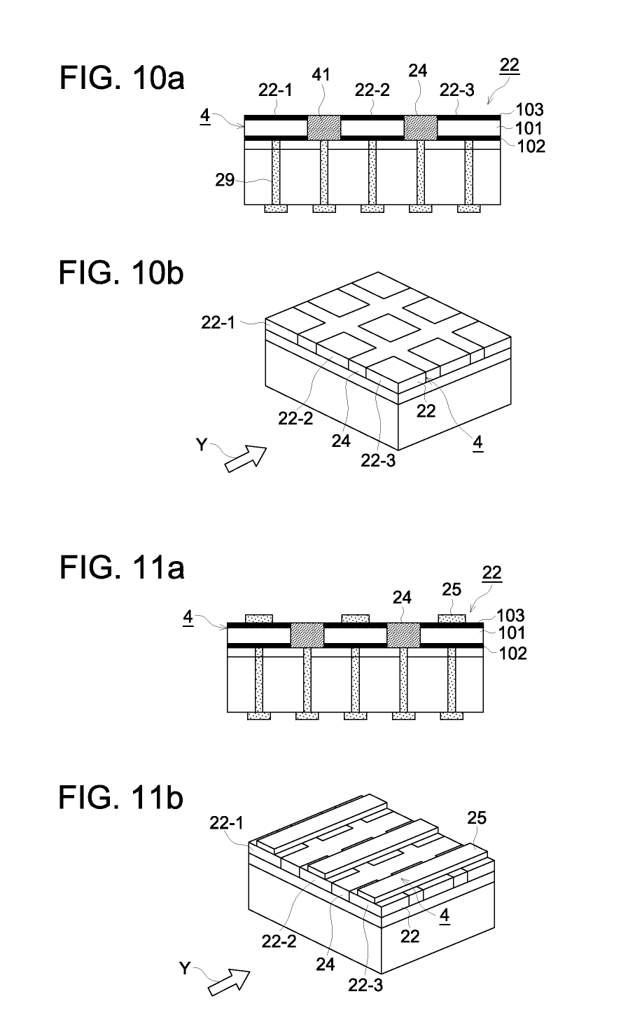

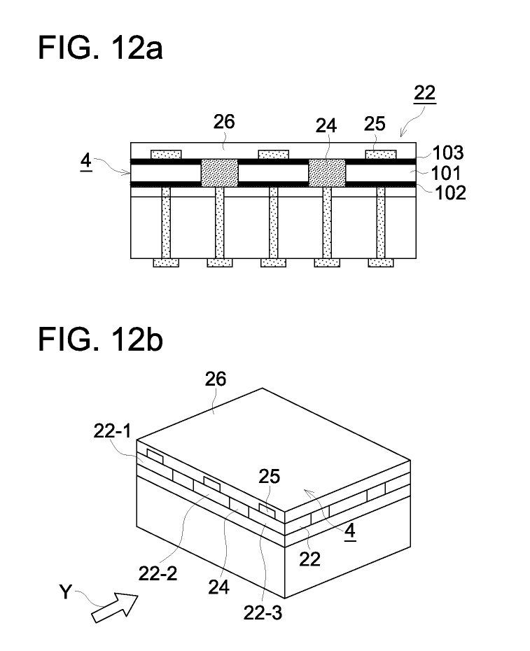

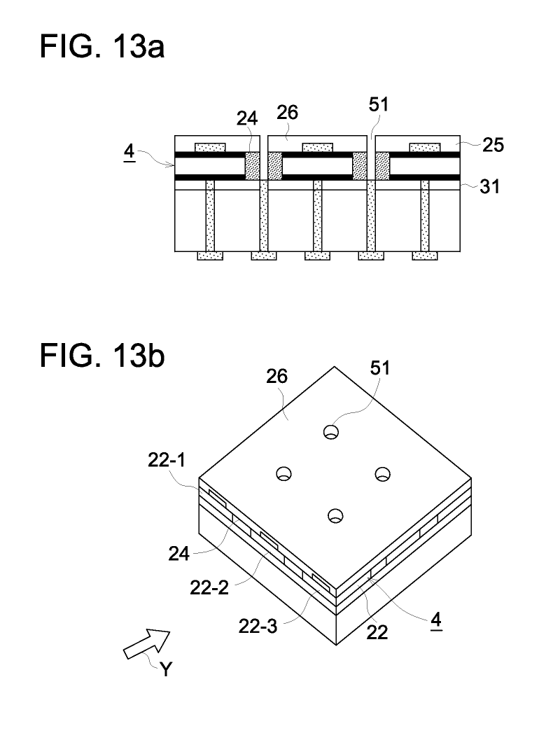

FIG. 4 shows a preparation flowchart in a method of preparing ultrasonic probe 2; FIGS. 5a, 5b, 5c and 5d each show a schematic diagram in a method of preparing organic piezoelectric element array 5; FIGS. 6a and 6b each show a schematic diagram of acoustic damping member 23; FIGS. 7a and 7b each show a schematic diagram of acoustic matching layer 31; FIGS. 8a and 8b each show a schematic diagram of a work body in which flat plate-shaped double-sided electrodes-providing inorganic piezoelectric element 50 provided on both surfaces of the inorganic piezoelectric element is installed; FIGS. 9a and 9b each show a schematic diagram of a work body in which inorganic piezoelectric element 22 is arrayed; FIGS. 10a and 10b each show a schematic diagram of a work body in which acoustic separation section 24 is formed; FIGS. 11a and 11b each show a schematic diagram of a work body in which common ground electrode 25 is formed; FIGS. 12a and 12b each show a schematic diagram of a work body in which acoustic matching layer 26 is formed; and FIGS. 13a and 13b each show a schematic diagram of a bored work body.

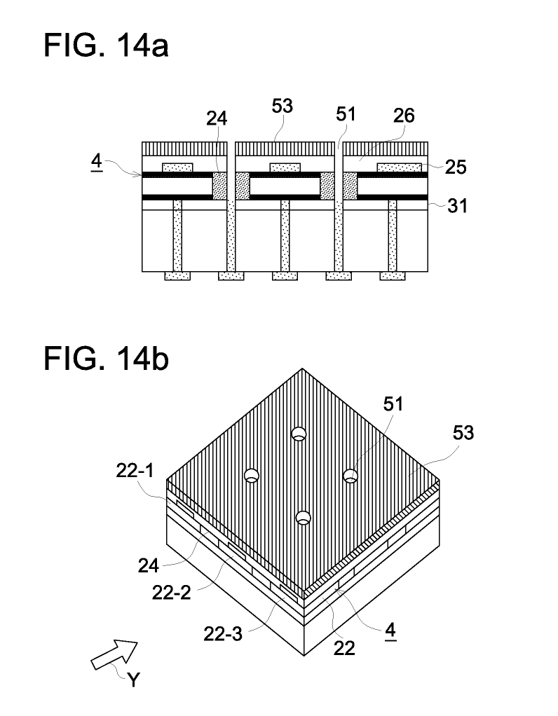

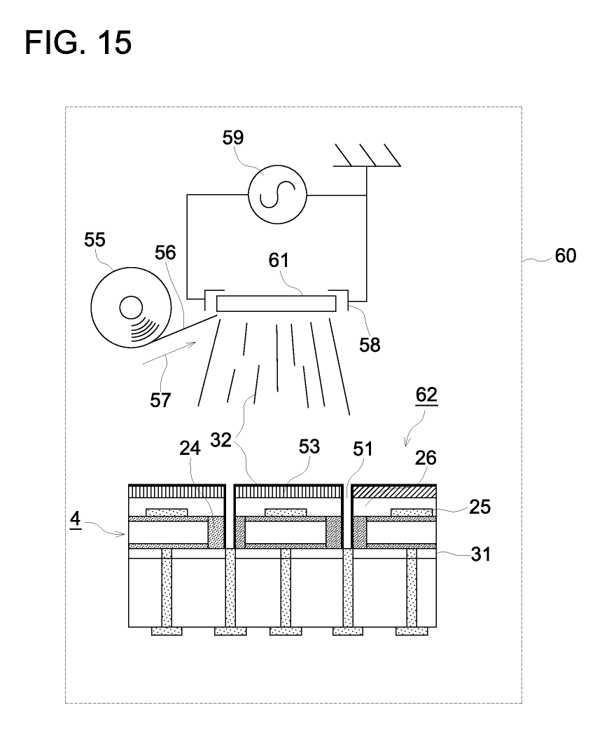

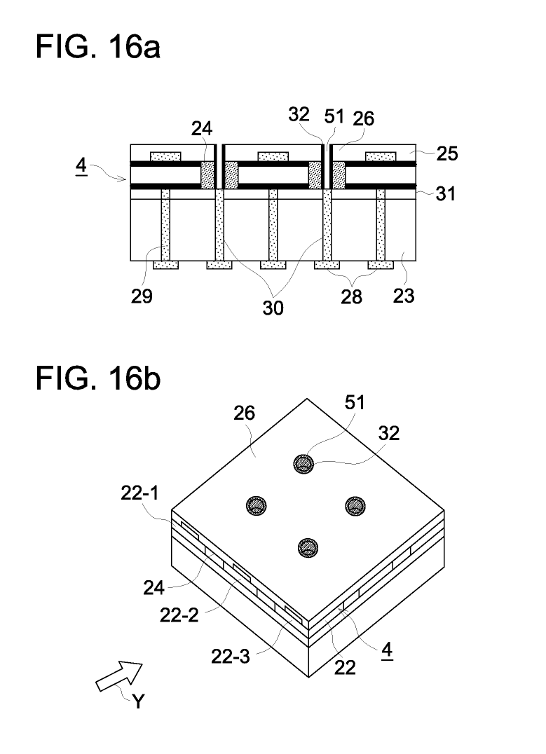

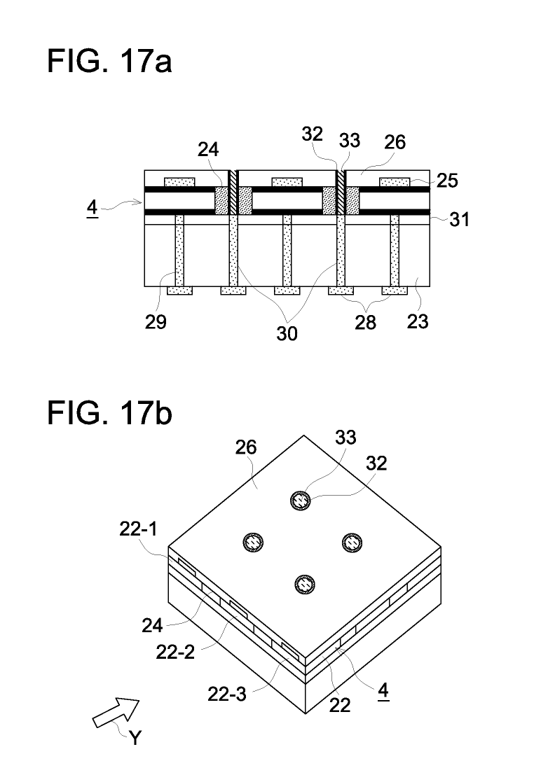

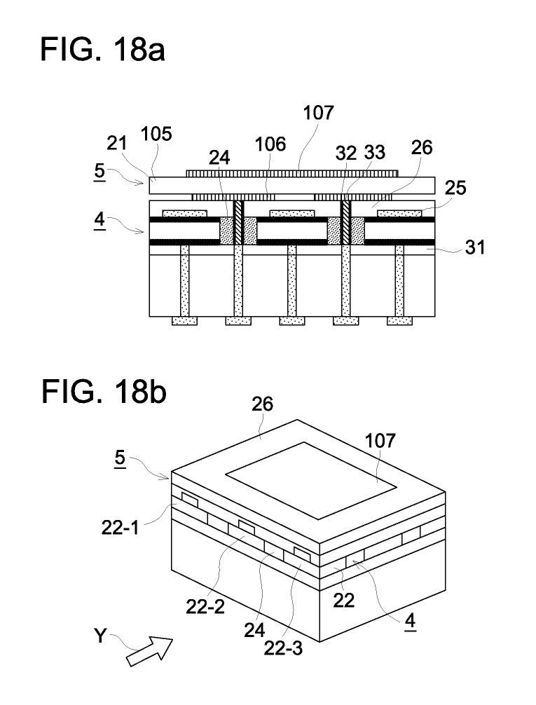

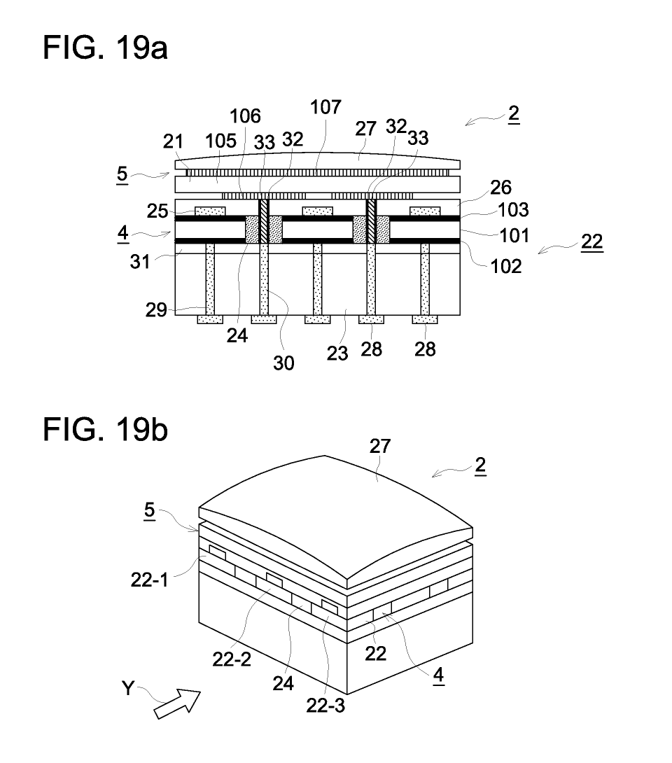

FIGS. 14a and 14b each show a schematic diagram of a work body in which photoresist is formed; FIG. 15 shows a schematic diagram of a process by which metal constituting conductive member 32 is evaporated on the work body; FIGS. 16a and 16b each show a schematic diagram of a work body from which the metal constituting conductive member 32 on photoresist, together with the photoresist, is removed; FIGS. 17a and 17h each show a schematic diagram of a work body in which core material 33 is formed in laser processing hole 51; FIGS. 18a and 18b each show a schematic diagram of a work body possessing a layered organic piezoelectric element array, and FIGS. 19a and 19b each show a schematic diagram of a work body in which acoustic matching layer 27 is formed.

In addition, as to FIG. 6a through FIG. 19b, FIGS. 6a, 7a, 8a, 9a, 10a, 11a, 12a, 13a, 14a, 16a, 17a, 18a and 19a each are a cross-sectional diagram of a work body prepared in each step, and FIGS. 6b, 7b, 8b, 9b, 10b, 11b, 12b, 13b, 14b, 16b, 17b, 18b and 19b each are an oblique perspective diagram. FIGS. 6a, 7a, 8a, 9a, 10a, 11a, 12a, 13a, 14a, 16a, 17a, 18a and 19a each are a diagram observed in the Y direction in each of FIGS. 6b, 7b, 8b, 9b, 10b, 11b, 12b, 13b, 14b, 16b, 17h, 18b and 19b.

Next, a method of preparing ultrasonic probe 2 will be described referring to a preparation flowchart in FIG. 4.

First, organic piezoelectric element array (the second piezoelectric element array) 5 is prepared in Step S10. As shown in FIG. 5a, provided is piezoelectric element 105 made of an organic piezoelectric material in the form of a flat plate, which has the predetermined thickness. Thickness of piezoelectric element 105 is appropriately designed to be set depending on frequency of ultrasonic waves to be received, kinds of organic piezoelectric materials and so forth, but for example, when receiving ultrasonic waves at a center frequency of 8 MHz, piezoelectric element 105 has a thickness of about 50 μm.

As the organic piezoelectric material, a vinylidene fluoride polymer is usable, for example. A vinylidene fluoride (VDF) based copolymer is also usable as the organic piezoelectric material, for example. This vinylidene fluoride based copolymer is a copolymer of vinylidene fluoride and another monomer, and usable examples of the foregoing another monomer include trifluoroethylene, tetrafluoroethylene, perfluoroalkylvinylether (PFA), perfluoroalkoxyethylene (PAE), perfluorohexaethylene and so forth. Since a vinylidene fluoride based copolymer varies an electromechanical coupling factor (piezoelectric effect) in the thickness direction, depending on the copolymerization ratio, an appropriate copolymerization ratio is employed in accordance with the specification of an ultrasonic probe or the like. In the case of a copolymer of vinylidene fluoride and trifluoroethylene, the vinylidene fluoride preferably has a copolymerization ratio of 60-99 mol %, and in the case of a composite element in which an organic piezoelectric element is layered on an inorganic piezoelectric element, the vinylidene fluoride more preferably has a copolymerization ratio of 85-99 mol %. In the case of such a composite element, preferable examples of the foregoing another monomer include perfluoroalkylvinylether (PFA), perfluoroalkylethylene (PAE) and perfluorohexaethylene. Further, polyurea, for example, is usable for the organic piezoelectric material. In the case of this polyurea, piezoelectric element 105 is preferably prepared via an evaporation polymerization method. An H₂N—R—NH₂ structure as a formula can be provide as a monomer for polyurea, wherein R may possess an alkylene group, a phenylene group a divalent heterocyclic group, or a heterocyclic group substituted by an arbitrary substituent. The polyurea may be a copolymer of a urea derivative and another monomer. As a preferred polyurea, provided is an aromatic polyurea obtained by using 4,4′-diaminodiphenylmethane (MDA) and 4,4′-diphenylmethanediisocyanate (MDI).

Next, as shown in FIG. 5b, plural electrodes 106 (106-11 and 106-12) separated to each other are formed on one main surface of piezoelectric element 105 made of this organic piezoelectric material by screen printing, evaporation, sputtering or the like, for example. These plural electrodes 106 may be formed in the two directions which are linearly independent in planar view, for example, so as to be arrayed in the form of a two-dimensional array placed in m rows and n columns in the two directions which are at right angles to each other (each of m and n represents a positive integer). Electrode 106 is in the form of a rectangle in planar view, for example, and size thereof is appropriately designed to be provided depending on resolution and so forth, for example, but a size of about 0.1 mm×0.1 mm is designed to be provided, for example.

In addition, in the present specification, when showing collective designation, a referential symbol in which a suffix is omitted is used, and when showing individual configuration, a referential symbol in which a suffix is provided is used.

Then, as shown in FIGS. 5c and FIG. 5d, electrode 107 is formed on the other main surface of piezoelectric element 105 made of this organic piezoelectric material via screen printing, evaporation, sputtering or the like. By doing this, formed is organic piezoelectric array (the second piezoelectric element array) 5 possessing plural electrodes 106 arranged in the form of an army two-dimensionally placed in m rows and n columns, which are provided on one main surface, and electrode 107 which is provided on the other main surface.

As to organic piezoelectric element 21 having such a structure, one piezoelectric element is constituted from each electrode 106, electrode 107 facing this each electrode, and piezoelectric element 105 made of an organic piezoelectric material present between each electrode 106 and electrode 107, resulting in appearance of inclusion of plural piezoelectric elements.

In the case of a method of preparing ultrasonic probe 2 in the present embodiment in such a way, plural piezoelectric elements are formed by forming plural electrodes 106 separated to each other on the surface of sheet-shaped piezoelectric element 105 made of an organic piezoelectric material. For this reason, no step of forming grooves with respect to sheet-shaped piezoelectric element 105 is conducted in order to form plural piezoelectric elements. Accordingly, in the case of ultrasonic probe 2 having such a structure, since no step of forming grooves with respect to organic piezoelectric element 21 is carried out, whereby a method of preparing organic piezoelectric element 21 is largely simplified, and it becomes possible to prepare ultrasonic probe 2 with reduced man-hours.

In addition, in those described above, plural electrodes 106 are formed on one main surface of piezoelectric element 105, and electrode 107 is subsequently formed on the other surface of piezoelectric element 105, but electrode 106 is formed on the other main surface of piezoelectric element 105, and plural electrodes 107 may be subsequently formed on one main surface thereof of piezoelectric element 105.

Next, as shown in FIGS. 6a and 6b, acoustic damping member 23 suitable for size of organic piezoelectric element 21 is provided in Step 11. Acoustic damping member 23 is equipped with a flat plate-shaped ultrasonic absorber to absorb ultrasonic waves, and absorbs ultrasonic waves radiating from the surface brought into contact with acoustic damping member 23 in inorganic piezoelectric element 22.

Acoustic damping member 23 has acoustic impedance nearly equal to that of the after-mentioned signal line S, whereby not only reflection of ultrasonic waves at the interface between acoustic damping member 23 and signal line S is inhibited, but also generation of acoustic crosstalk with respect to inorganic piezoelectric element 22 is inhibited. Acoustic damping member 23 is formed of silicon rubber, for example.

Next, as shown in FIGS. 7a and 7b, acoustic matching layer 31 is formed on the main surface of acoustic damping member 23 in Step S12 to form signal line S(S1, S2, S3, S4, S5 and so forth) by passing through acoustic damping member 23 and acoustic matching layer 31, and conductive pad 28 is formed on the surface on the opposite side of acoustic member 23 of signal line S. This signal line S is connected to electrodes of inorganic piezoelectric element 22 and organic piezoelectric element 21 to be layered in the postprocessing step.

Next, inorganic piezoelectric elements 22 are arranged and prepared in Step S13. First, flat plate-shaped double-sided electrodes-providing inorganic piezoelectric element 50 is layered on acoustic matching layer 31. The double-sided electrodes-providing inorganic piezoelectric element 50 is obtained by forming an electrode on each of both surfaces of an inorganic piezoelectric plate. The same method as a method of forming an electrode for the organic piezoelectric element is utilized for a method of forming the electrode. For example, it is formed via screen printing, evaporation, sputtering or the like.

When the double-sided electrode inorganic piezoelectric element 50 is layered on acoustic matching layer 31, signal lines S1 through S5 are electrically connected to an electrode formed on one main surface of the double-sided electrode inorganic piezoelectric element 50.

Examples of materials for the inorganic piezoelectric plate include PZT quartz, lithium niobate (LiNbO₃), potassium tantalate niobate {K (Ta, Nb) O₃}, barium titanate (BaTiO₃), lithium tantalate (LiTaO₃), strontium titanate (SrTiO₃), and so forth.

Next, as shown in FIGS. 9a and 9b, grooves 41 are formed in the layering direction in the double-sided electrodes-providing inorganic piezoelectric element 50 employing a dicing saw or the like, for example to array them, until acoustic matching layer 31 appears exposed. Constituted is the first piezoelectric element array 4 possessing plural inorganic piezoelectric elements 22 (22-1, 22-2, 22-3 and so forth) arranged in the form of a two-dimensional array placed in p rows and q columns in the two directions which are at right angles to each other (each of p and q represents a positive integer), and plural grooves 41 are formed in the two directions.

Electrode surfaces in the double-sided electrodes-providing inorganic piezoelectric element are divided to each other by forming grooves 41. The upper electrode surface is divided into electrodes 103, and the lower electrode surface is divided into electrodes 102. Size thereof is appropriately designed to be provided depending on resolution and so forth, for example, but a size of about 0.4 mm×0.4 mm is designed to be provided, for example. Each electrode 102 is connected to each of signal lines S1, S3 and S5 to supply electrical signals from the outside via conductive pad 28.

Next, as shown in FIGS. 10a and 10b, in order to avoid acoustic crosstalk and resonance of each inorganic piezoelectric element 22, a member having different acoustic impedance from that of each inorganic piezoelectric element 22, for example, an acoustic separation material made of a low acoustic impedance resin or the like is filled in grooves 41 to form acoustic separation section 24 in Step S14. For example, thermosetting resins such as a polyimide resin, an epoxy resin and so forth are usable as such a resin.

Next, as shown in FIGS. 11a and 11b, commonly grounding electrode 25 to be commonly grounded is formed in the form of a layer on the upper surface of the first piezoelectric element array 4 via screen printing, evaporation, sputtering or the like, for example, in Step S15. This commonly grounding electrode 25 is grounded via unshown wiring.

Next, as shown in FIGS. 12a and 12b, acoustic matching layer 26 is formed from the upper portion of commonly grounding electrode 25 in Step S16.

Next, as shown in FIGS. 13a and 13b, conducted is a hole-boring operation to form the second signal line 30 connected to organic piezoelectric element 21 before placing organic piezoelectric element 21 prepared as described above on acoustic matching layer 26 in Step S17. As previously described, the second signal line 30 is formed on the upper surface of acoustic matching layer 31, and the cross-section of the second signal line 30 appears exposed. A part of acoustic matching layer 26 with acoustic separation section 24 up to signal line S from the upper surface of acoustic matching layer 26 is removed and bored to form through-holes.

For the hole-boring operation to form through-holes by removing a part of acoustic matching layer 26 and acoustic separation section 24, laser or a drill is used, for example. When using laser, if UV laser such as excimer laser or the like is employed, acoustic matching layer 26 and acoustic separation section 24 are clearly removed via ablation processing, and laser-processing hole 51 can be obtained. Since depth of laser-processing hole 51 can be controlled by laser exposure duration, laser-processing hole 51 having appropriate depth can be formed. In addition, acoustic matching layer 26 and acoustic separation section 24 are sublimated and can be also removed via thermal processing with high-power laser such as carbon dioxide laser or the like.

Laser-processing hole 51 is designed to set a diameter of the second signal line 30. The diameter of the second signal line is desired to be a diameter which does not shield ultrasonic waves in consideration of processing simplicity, and is, for example, preferably 50-60 μm.

Further, when using a drill, hole-boring is carried out while monitoring whether or not the drill goes through signal line S. Since it can be detected that a tip of the drill reaches signal line S, extra hole-boring can be avoided.

Next, as shown in FIGS. 14a and 14b, photoresist film 53 is formed in Step S18. A coater such as a spinner or the like is employed for coating the photoresist. Coating should be carefully conducted in such a way that the photoresist is not penetrated into laser-processing hole 51.

Next, conductive member 32 is formed on the laser-processing hole 51 inner circumferential surface in Step S19. FIG. 15 is a schematic diagram showing film-formation of a conductive member employing an evaporator. Board 61, electrode 58 fitted with board 61, high-frequency power supply 59 as a thermal source for board 61, and metal roll 55 in the form of a roll obtained by rolling metal plate 56 are placed in the upper part of evaporation chamber 60. Metal plate 56 is transferred onto board 61 from metal roll 55 in the direction of arrow 57. Metal constituting metal plate 56 is, for example, aluminum, gold or the like.

Work body 62 after preparation steps up to Step S18 is grounded to the lower direction of board 61 to conduct evaporation. Not only conductive member 32 as a conductive layer made of metal is formed on the laser-processing hole 51 inner circumferential surface, but also a metal layer is formed on photoresist film 53.

As previously described, conductive member 32 preferably has a thickness of about 0.05 μm.

Next, in Step S20, removal of the photoresist film is made employing a solution or the like. FIG. 16 is a schematic diagram showing a work body in which the metal film on a photoresist is removed via removal of the photoresist.

Next, a low acoustic impedance member as a material for core material 33 is filled in the laser-processing hole 51 inner spacing and solidified. Conductive member 32 appears to be thinly coated on the outer circumferential surface of core material 33. FIGS. 17a and 17b each is a diagram showing a work body in which a low acoustic impedance member is filled in laser-processing hole 51, and solidified to form core material 33. Formed is a part of the signal line to reach signal line S after passing through acoustic matching layer 26 and acoustic separation section 24. A low acoustic impedance conductive member having the same acoustic impedance as that of acoustic separation section 24 is employed for core material 33 to avoid acoustic crosstalk.

Specifically, each material is selected in such a way that acoustic impedance of acoustic separation section 24 and acoustic impedance of core material 33 satisfy the following formula (1);

|Z1−Z2|≦0.5·10⁶ kg/m²·s (1),

wherein Z1 represents the acoustic impedance of acoustic separation section 24, and Z2 represents the acoustic impedance of core material 33. Reflection of ultrasonic waves at the portion between acoustic separation section 24 and core material 33 can be largely reduced by selecting material satisfying Formula (1).

PZT is conventionally used as an inorganic piezoelectric material, and PZT has an acoustic impedance of 29·10⁶ kg/m²·s to about 38·10⁶ kg/m²·s. Material having an acoustic impedance value largely different from that of PZT, for example, silicon rubber is utilized for acoustic separation section 24 provided between inorganic piezoelectric elements 22 to each other so as not to transmit ultrasonic waves at the interface with PZT. The silicon rubber roughly has an acoustic impedance of 0.99·10⁶ kg/m²·s to 1.46·10⁶ kg/m²·s, which is largely different from that of PZT. Accordingly, ultrasonic waves generated inside PZT are mostly reflected at the interface between PZT and the silicon rubber.

Material having an acoustic impedance nearly equal to another impedance of acoustic separation section 24 is selected as core material 33.

Usable examples of the specific material include aluminum, aluminum alloys (for example, Al—Mg alloy), magnesium alloys, Macor glass, glass, fused quartz, copper graphite, polyethylene (PE) or polypropylene (PP), polycarbonate (PC), ABC resin, polyphenylene ether (PPE), ABS resin, AAS resin, AES resin, nylon (PA6 or PA6-6), PPO (polyphenyleneoxide), PPS (polyphenylenesulfide: those containing glass fibers are also allowed), PPE (polyphenylene ether), PEEK (polyetherether ketone), PAI (polyamide imide), PETP (polyethylene terephthalate), PC (polycarbonate), epoxy resin, urethane resin, silicone resin and so forth.

Preferably usable are those molded by containing zinc oxide, titanium oxide, silica or aluminum, colcothar, ferrite, tungsten oxide, ytterbium oxide, barium sulfate, tungsten, molybdenum, other metal oxides, or the like as a filler in epoxy resin, urethane resin or silicone resin.

For example, when core material 33 is composed of metal oxide and silicone resin, the silicone resin possesses plural silicone bonds as Si—O bonds.

Those having dimethyl polysiloxane as a main component are preferable as silicone resins, and usable are those preferably having a polymerization degree of 3000-10000.

Preferably usable are those further containing a silicone compound represented by the following Formula (1).

R¹(R¹₂SiO)_{X}(R¹R²SiO)_{Y}SiR¹₃ Formula (1),

where R¹ represents a monovalent hydrocarbon group or hydrogen atom; R² represents an alkyl group or polyether group; X is an integer of at least 0; and Y is an integer of at least 1.

These are commercially available, and usable examples thereof include those produced by Shin-Etsu Chemical Co., Ltd., for example, KE742U, KE752U, KE931U, KE941U, KE951U, KE961U, KE850U, KE555U, KE575U and so forth; those produced by Momentive Performance Materials Inc., for example, TSE221-3U, TSE221-4U, TSE2233U, XE20-523-4U, TSE27-4U, TSE260-3U and TSE-260-4U; and those produced by Dow Corning Toray Co., Ltd., for example, SH35U, SH55UA, SH831U, SE6749U, SE1120U, SE4704U and so forth.

The silicone resin preferably has a content of 40% by weight or more, and more preferably has a content of 40-80% by weight or more, based on the weight occupied by core material 33, in view of acoustic characteristics and durability.

Examples of metal oxide particles usable in the present invention include TiO₂, SnO₂, ZnO, Bi₂O₃, WO₃, ZrO₂, Fe₂O₃, MnO₂, Y₂O₃, MgO, BaO and Yb₂O₃. Of these, ZnO, TiO₂ and Yb₂O₃ are preferably utilized in view of acoustic characteristics.

Metal oxide particles preferably have an average particle diameter of 1-200 nm, and more preferably have an average particle diameter of 5-20 nm. Each of 100 particle diameters was measured to determine an average particle diameter, and the average particle diameter is a value obtained by determining a number average of these values. The particle diameter is a mean value obtained from the maximum diameter and the minimum diameter of particles, which are measured from an image observed by an electron microscope.

Metal oxide particles preferably have a content of 10-60% by weight, and more preferably have a content of 15-50% by weight in view of acoustic characteristics.

Metal oxide particles are commercially available. Examples of ZnO include grade 1 zinc oxide, fine zinc oxide, FINEX-30, FINEX-30SLP2, FINEX-30WLP2, FINEX-50, FINEX-50SLP2, FINEX-50WLP2, NANOFINE-50, NANOFINE-50A and NANOFINE-50SD produced by Sakai Chemical Industry Co., Ltd.; Zinc oxide for pharmaceutical use, ZINCOX, Super F-1, Super F-2 and Super F-3, produced by Hakusuitech Ltd.; MAXLIGHT ZS-64 (silica-coating zinc oxide) produced by SHOWA DENKO K.K., and so forth. Examples of TiO₂ include R-45M, R32, R-11P, R-21, D-918, STR-60C-LP, STR-100C-LP and STR-100A-LP produced by Sakai Chemical Industry Co., Ltd.; R-820, R-830 and R-670, produced by Ishihara Sangyo Ltd.; MAXLIGHT TS-043 (silica-coating titanium oxide) produced by SHOWA DENKO K.K.; and so forth. As Yb₂O₃, high purity Yb₂O₃ particles produced by Shin-Etsu Chemical Co., Ltd. are usable.

Core material 33 may be those obtained by mixing and kneading a silicone resin and silica-coating oxide particles, and by adding a vulcanizing agent into the kneaded to conduct vulcanization-molding.

Further, secondary vulcanization can be carried out, if desired. Usable examples of vulcanizing agents include peroxide based vulcanizing agents such as 2,5-dimethyl-2,5-ditert-butylperoxyhexane, p-methylbenzoylperoxide, ditert-butylperoxide, and so forth. An amount of the peroxide based vulcanizing agent is preferably 0.3-2% by weight, based on silicone rubber in the acoustic lens composition. Further, vulcanizing agents other than the peroxide based vulcanizing agents may be used.

When mixing a silicone resin and silica-coating oxide particles, water content is preferably removed from metal oxide particles. Temperature of the vulcanization-molding is preferably 100-200° C.

Further, the following additives may be contained in an amount of approximately 5% by weight. Examples thereof include titanium oxide, alumina, cerium oxide, iron oxide, barium sulfate, organic fillers, coloring pigments, and so forth.

As described above, reflection of ultrasonic waves between acoustic separation section 24 and core material 33 can be reduced by matching an impedance of core material 33 and another impedance of acoustic separation section 24

In order to fill core material 33 in laser-processing hole 51, a needle of a dispenser filled in by a core material is approached to a bored hole, and the core material is pressed and filled in by an air pressure.

Next, as shown in FIGS. 18a and 18b, sheet-shaped organic piezoelectric element array (the second piezoelectric element array) 5 prepared in another step as described above is layered on acoustic matching layer 26 in Step S22. Organic piezoelectric element array 5 is fixed on inorganic piezoelectric element 22 via adhesion. Electrode 106 fitted with organic piezoelectric element 21 is layered so as to correspond to the second signal line 30. Electrode 107 fitted with organic piezoelectric element 21 is a common electrode, and grounded through unshown wiring.

Next, as shown in FIGS. 19a and 19b, acoustic matching layer 27 is formed on organic piezoelectric element 21 in Step S23. Acoustic matching layer 27 is composed of a single layer or plural layers, if desired. In order to widely expand the reception frequency range, acoustic matching layer 27 is preferably composed of plural layers.

With that, ultrasonic probe 2 having a structure as shown in FIG. 3 is prepared, and a preparation flow of ultrasonic probe 2 is terminated.

In the present embodiment, inorganic piezoelectric element 22 possesses a single layer as piezoelectric element 101 fitted with electrode 102 and electrode 103, respectively, provided on the both surfaces of piezoelectric element 101, but may possess plural layers layered as piezoelectric element 101 fitted with electrode 102 and electrode 103, respectively, provided on the both surfaces of piezoelectric element 101.

Further, in the present embodiment, organic piezoelectric element 21 possesses a single layer as piezoelectric element 105 fitted with electrode 106 and electrode 107, respectively, provided on the both surfaces of piezoelectric element 105, but may possess plural layers formed as piezoelectric element 105 fitted with electrode 106 and electrode 107, respectively, provided on the both surfaces of piezoelectric element 105. When transmitting ultrasonic waves, power thereof is possible to be increased, and when receiving ultrasonic waves, reception sensitivity thereof is possible to be improved by forming the plural layers.

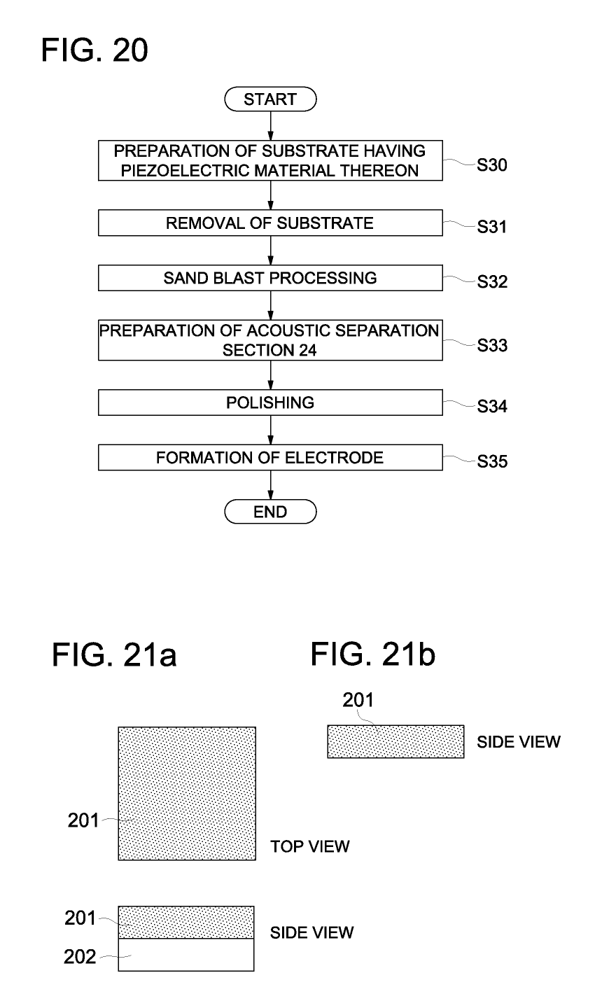

Further, as described in FIGS. 8a and 8b, there is a method of preparing the first piezoelectric element array 4 provided after layering flat plate-shaped both-sided electrodes-providing inorganic piezoelectric element 50 on acoustic matching layer 31. In addition, there is another method of layering it on acoustic matching layer 31 after preparing the first piezoelectric element array 4 in advance. High-volume production of the first piezoelectric element array 4 is improved by separately preparing the first piezoelectric element array 4. The method of preparing the first piezoelectric element array 4 in advance will be described referring to FIG. 20, FIGS. 21a and 21b, FIGS. 22a and 22b, FIGS. 23a and 23b, and FIGS. 24a and 24b.

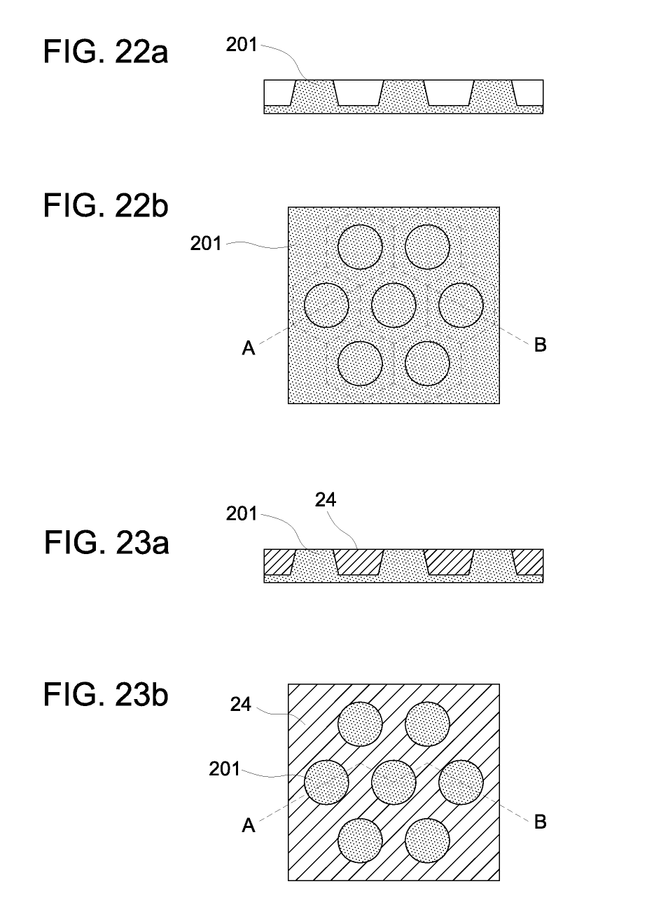

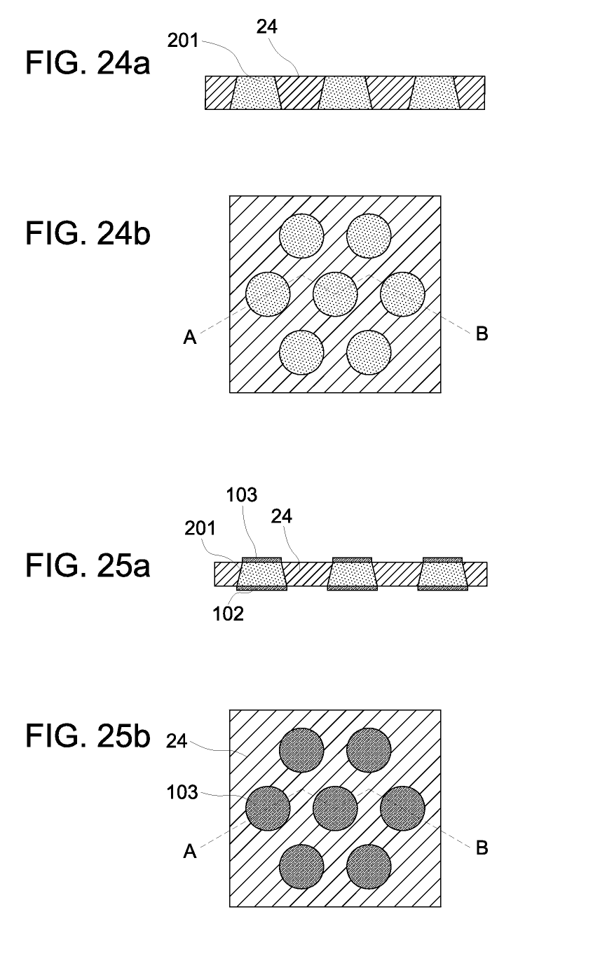

FIG. 20 shows a preparation flowchart for a method of preparing the first piezoelectric element array 4. FIGS. 21a and 21b each show a schematic diagram of a substrate having a piezoelectric material thereon. FIGS. 22a and 22b each show a schematic diagram of a work body having been subjected to sandblast processing. FIGS. 23a and 23b each show a schematic diagram of a work body in which acoustic separation section 24 is formed. FIGS. 24a and 24b each show a schematic diagram of a work body in which the acoustic separation section is exposed onto both surfaces of the acoustic separation section via polishing. FIGS. 25a and 25b each show a schematic diagram of a work body in which electrodes 102 and 103 are formed on both surfaces of PZT 201.

Next, a method of preparing the first piezoelectric element array 4 is described based on a preparation flowchart of FIG. 20. First, a substrate fitted with a piezoelectric material as shown in FIG. 21a is prepared in Step S30. PZT or the like is used as an inorganic piezoelectric material as described before. PZT is coated on substrate 202, and then calcined to obtain PZT 201 calcined on substrate 202.

Next, substrate 202 is removed via polishing in Step S31 to obtain a substrate of calcined PZT 201 as shown in FIG. 21b.

Next, sandblast processing is carried out to emboss the shape of each inorganic piezoelectric element 22 constituting the first piezoelectric element array 4 in Step S32. FIGS. 22a and 22b show a schematic diagram of a work body having been subjected to the sandblast processing. FIG. 22b shows a top view, and FIG. 22a shows a cross-sectional view along a zig-zag dashed line AB, based on FIG. 22b (hereinafter, the same manner up to FIGS. 25a and 25b from FIGS. 23a and 23b). An array of inorganic piezoelectric elements 22 may be in the form of a grid pattern as shown in FIG. 9, but may be composed of a honey comb structure as shown in FIG. 22b. A feature of sandblast processing makes etching portions to be slightly in the form of an earthenware mortar, but there appears no problem in view of fulfillment of a function of an ultrasonic probe,

Next, acoustic separation section 24 is prepared in Step S33. Specifically, a material having an impedance nearly equal to that of the material used as core material 33 described above is selected to embed it in etching portions of a PZT 201 substrate as shown in FIG. 23. In cases where silicon rubber is employed as core material 33, silicon rubber having the same composition as that of the core material may be used for acoustic separation section 24, and silicon rubber having a different composition from that of the core material may be selected in consideration of preparation-related matters. Further, an epoxy resin may be employed as described above.

Next, as to a work body prepared in Step S33, one surface of a work body is subjected to polishing in order to separate the first piezoelectric element army 4 into each of the resulting inorganic piezoelectric elements 22 by exposing acoustic separation section 24 on the front surface and back surface of the work body in Step S34. FIGS. 24a and 24b show an outline of the work body after polishing.

Finally, electrodes 102 and 103 are formed on the surfaces on which PZT 201 is exposed as shown in FIGS. 25a and 25b to complete the first piezoelectric element array 4 in Step S35.

As described above, in the present embodiment, generation of acoustic crosstalk between plural inorganic piezoelectric elements 22 can be avoided by forming an acoustic separation section provided between the first piezoelectric elements to each other, arrayed in the first piezoelectric element array, and by utilizing a core material having an acoustic impedance nearly equal to another acoustic impedance of the acoustic separation section, the core material connected to the second electrode by passing through the acoustic separation section, and a signal line possessing a conductive layer coated on an outer circumferential surface of the core material.

Further, the number of electrodes 106 of organic piezoelectric element 21 and the number of inorganic piezoelectric elements 22 are possible to be designed in accordance with the specification desired for inorganic piezoelectric elements 22 and organic piezoelectric element 21 by making the number of electrodes 106 of organic piezoelectric element 21 (the number of organic piezoelectric elements 21) to be different from the number of inorganic piezoelectric elements 22. For example, it is possible to reduce the area of each piezoelectric element when the number of piezoelectric elements possessed by organic piezoelectric element 21 is increased, whereby in cases where organic piezoelectric element 21 is used for receiving signals, reception resolution thereof can be improved.

Further, the area of electrode 106 of organic piezoelectric element 21 and the area of electrode 102 of inorganic piezoelectric element 22 in addition to the area of electrode 103 of inorganic piezoelectric element 22 can be designed in accordance with the specifications specified for the inorganic piezoelectric element 22 and the organic piezoelectric element 21 by making the area of electrode 106 of organic piezoelectric element 21 to be different from the area of electrode 102 of inorganic piezoelectric element 22 or the area of electrode 103 of inorganic piezoelectric element 22. The total area of grooves each between inorganic piezoelectric elements 22 is designed to be small by making the area of, for example, one inorganic piezoelectric element 22, and similarly, transmission power of ultrasonic probe 2 can be increased by increasing the total area obtained via addition of the area of each of inorganic piezoelectric elements 22.

Further, generation of acoustic crosstalk between the first piezoelectric elements arrayed in the first piezoelectric element array can be avoided by utilizing a method of preparing an ultrasonic probe comprising the steps of forming an acoustic separation section between the first piezoelectric elements to each other arrayed in the first piezoelectric element array; and filling a core material having an acoustic impedance nearly equal to another acoustic impedance of the acoustic separation section in a through-hole inner spacing for solidification after forming a through-hole in the acoustic separation section to form a conductive layer on the circumferential surface of the through-hole.

### EXPLANATION OF NUMERALS

1 Ultrasonic diagnosis apparatus
2 Ultrasonic probe
3 Cable
4 The first piezoelectric element array
5 Organic piezoelectric element array
11 Operation input section
12 Transmission circuit
13 Reception circuit
14 Image processing section
15 Display section
16 Control section
21 Organic piezoelectric element
22 inorganic piezoelectric element
23 Acoustic damping member
24 Acoustic separation section
25 Common ground electrode
26, 27, and 31 Acoustic matching layer
28 Conductive pad
29 The first signal line
30 The second signal line
32 Conductive member
33 Core material
41 Groove
50 Double-sided electrodes-providing inorganic piezoelectric element
51 Laser-processing hole
53 Photoresist film
55 Metal roll
56 Metal plate
58 Electride
59 High-frequency power supply
60 Evaporation chamber
61 Board
62 Work body
102, 103, 106, and 107 Electrode
202 Substrate

1. An ultrasonic probe comprising:
a first piezoelectric element array in which first piezoelectric elements each as a both-sided electrodes-providing piezoelectric element are two-dimensionally arrayed;
a second piezoelectric element array layered on the first piezoelectric element array, in which second piezoelectric elements are two-dimensionally arrayed, a first electrode is placed on a second piezoelectric element surface on an opposite side of the first piezoelectric element array, and a second electrode is placed on another second piezoelectric element surface on a side of the first piezoelectric element array,
an acoustic separation section provided between the first piezoelectric elements to each other, arrayed in the first piezoelectric element array, and
a signal line comprising a conductive layer coated on an outer circumferential surface of a core material and the core material having an acoustic impedance nearly equal to another acoustic impedance of the acoustic separation section, the signal line connected to the second electrode by passing through the acoustic separation section.

2. The ultrasonic probe of claim 1,
wherein an area of each of the first piezoelectric elements to which a voltage is applied is different from an area of each of the second piezoelectric elements to which a voltage is applied.

3. The ultrasonic probe of claim 1 or 2,
wherein the number of the first piezoelectric elements arrayed in the first piezoelectric element array is different from the number of the second piezoelectric elements arrayed in the second piezoelectric element array.

4. A method of preparing an ultrasonic probe, comprising the steps of:
forming a through-hole in an acoustic separation section provided between the first piezoelectric elements to each other in a first piezoelectric element array in which first piezoelectric elements each as a both-sided electrodes-providing piezoelectric element are two-dimensionally arrayed;
forming a conductive layer on a through-hole inner circumferential surface so as to form a through-hole inner spacing; and
filling a core material having an acoustic impedance nearly equal to another acoustic impedance of the acoustic separation section in the through-hole inner spacing.
